(19) United States
(12) Patent Application Publication
(10) Pub. No.: US 2017/0267947 A1
(43) Pub. Date: Sep. 21, 2017

(54) DETERGENT CONTAINING AT LEAST ONE LACCASE AS A DYE-TRANSFER INHIBITOR

(71) Applicant: Henkel AG & Co. KGaA, Duesseldorf (DE)

(72) Inventors: Nina Mussmann, Willich (DE); Timothy O'Connell, Landsberg am Lech (DE); Thomas Weber, Dormagen (DE); Mareile Job, Leverkusen (DE); Hendrik Hellmuth, Darmstadt (DE); Michael Strotz, Koeln (DE); Inken Prueser, Duesseldorf (DE)

(73) Assignee: Henkel AG & Co. KGaA, Duesseldorf (DE)

(21) Appl. No.: 15/313,386

(22) PCT Filed: May 27, 2015
(86) PCT No.: PCT/EP2015/061622

§ 371 Date: Nov. 22, 2016

(30) Foreign Application Priority Data
Jun. 5, 2014 (DE) 10 2014 210 791.1

(51) Int. Cl. C11D 3/00; C12N 9/02; C11D 11/00; C11D 3/386; C11D 3/37

(52) U.S. Cl. CPC C11D 3/0021; C11D 3/38654; C11D 3/3776; C11D 11/0017; C12N 9/0061; C12Y 110/03002

(57) ABSTRACT

The present disclosure relates to the use of specific laccases as dye transfer-inhibiting active substances during the washing of textiles, and to detergents containing said laccases.

### CROSS-REFERENCE TO RELATED APPLICATIONS

This application is a U.S. National-Stage entry under 35 U.S.C. §371 based on International Application No. PCT/EP2015/061622, filed May 27, 2015, which was published under PCT Article 21(2) and which claims priority to German Application No. 10 2014 210 791.1, filed Jun. 5, 2014, which are all hereby incorporated in their entirety by reference.

### TECHNICAL FIELD

This disclosure relates to the use of specific laccases as dye transfer-inhibiting active substances during the washing of textiles, and to detergents containing said laccases.

### BACKGROUND

Laccases (EC 1.10.3.2) are copper-containing “blue” enzymes that are found in many plants, fungi, and microorganisms. Laccases are oxidoreductases. The catalytically active center contains four copper ions, which can be differentiated according to the spectroscopic properties thereof. The “blue” type 1 copper is involved in the substrate oxidation, and one type 2 copper ion and two type 3 copper ions form a trinuclear cluster that binds oxygen and reduces to water. Laccases are also called p-diphenol oxidases. In addition to diphenols, laccases also oxidize many other substrates, such as methoxy-substituted phenols and diamines. As regards the substrates thereof, laccases are remarkably non-specific. Due to the broad substrate specificity thereof and ability to oxidize phenolic compounds, laccases have aroused considerable interest in industrial applications. The many promising areas for use of laccases include, for example, delignification and bonding of fiberboard in the wood industry, dyeing of substances and detoxification of dye wastewater in the textile industry, and use in biosensors.

With the aid of mediators—i.e., intermediary molecules—laccases can even oxidize substrates that otherwise could not be oxidized thereby. The mediators are typically “small molecule compounds” that are oxidized by laccases. The oxidized mediator then in turn oxidizes the actual substrate. The first laccase was discovered as early as 1883, in the Japanese lacquer tree (Rhus vernicifera). Laccases can be found in many plants such as peach, tomato, mango, and potato; laccases are even known to be present in certain insects. The most commonly used laccases, however, are derived from fungi, for example, from the species Agaricus, Aspergillus, Cerrena, Curvularia, Fusarium, Lentinius, Monocillium, Myceliophtora, Neurospora, Penicillium, Phanerochaete, Phlebia, Pleurotus, Podospora, Schizophyllum, Sporotrichum, Stagonospora, and Trametes.

In nature, the function of laccases lies, inter alia, in participation in breaking down lignocellulose, biosynthesis of cell walls, browning of fruits and vegetables, and prevention of microbial attacks on plants.

In addition to the substances that are indispensable for the washing process, such as surfactants and builder materials, detergents generally also contain additional components that can be collectively referred to as washing aids and include groups of active materials as varied as foaming regulators, graying inhibitors, bleaching agents, bleaching activators, and enzymes. Such aids also include substances (dye transfer inhibitors (DTIs)) that are intended to prevent dyed textiles from producing an altered color impression after washing. This altering of the color impression of washed—i.e., clean—textiles can be due, on one hand, to removal (“fading”) of dye portions from the textile through the washing process; on the other hand, dyes that have been released from differently-colored textiles may be deposited onto the textile (“discoloration”). The discoloration aspect may also play a role in undyed laundry articles if said articles are washed with dyed laundry articles. To avoid these unwanted side effects of the removal of dirt from textiles by treatment with typically surfactant-containing aqueous systems, detergents contain active substances that are intended to prevent the release of dyes from the textile or at least avoid deposition of released dyes present in the washing liquor onto textiles, especially if said detergents are provided as so-called color or color-safe detergents for washing colored textiles.

Many of the commonly used polymers, however, have such a high affinity for dyes that said polymers pull said dyes out from the dyed fibers with detrimentally increased intensity, thus leading to an increased loss of color.

For sustainable economy, it is also desirable to achieve a dye transfer-inhibiting effect not through (stoichiometric) binding but rather in a manner that allows for the use of lower amounts of active substances.

It is also known that dye transfer inhibitors often cause problems in liquid detergent formulations, in particular because optical brighteners and DTIs in an aqueous detergent matrix are not compatible with a conventional composition. Thus, simultaneously incorporating an optical brightener and a polymeric dye transfer inhibitor in a liquid detergent matrix immediately leads to increased turbidity and subsequent phase separation.

This is especially disadvantageous if the liquid detergent or cleaning agent is intended to be clear and transparent, or at least translucent, and also is intended to be distributed in transparent/translucent packaging, for example, from the aesthetic point of view.

The present invention therefore addresses the problem of providing a suitable dye transfer inhibitor that prevents, or at least reduces, the disadvantages known in the prior art, and can be used in both solid and aqueous detergent formulations.

### BRIEF SUMMARY

Detergents and methods for washing dyed textiles using the detergents are provided herein. In one embodiment, the detergent includes at least one laccase as a dye transfer inhibitor.

In another embodiment, the detergent includes at least one laccase as a dye transfer inhibitor. the at least one laccase has a redox potential under 460 mV. The standard redox potential of the laccase is defined as the potential of the T1 copper center. The at least one laccase is selected from laccases that have the consensus sequence HCHx(3)Hx(4)M, wherein x stands for “any amino acid” and the number in parentheses that follows the x sets forth the number of said “amino acid(s)”.

In yet another embodiment, the method includes the step of applying a detergent comprising at least one laccase as a dye transfer inhibitor to a dyed textile.

### DETAILED DESCRIPTION

The following detailed description is merely exemplary in nature and is not intended to limit the invention or the application and uses of the invention. Furthermore, there is not intention to be bound by any theory presented in the preceding background or the following detained description.

It is contemplated herein to provide specific laccases that have low redox potential, as dye transfer inhibitors in detergents.

A first aspect of the present disclosure is therefore a detergent that contains at least one laccase as a dye transfer inhibitor.

As contemplated herein, preferred laccases are those from fungi, plants, and—in particular—bacteria that have a low redox, wherein the standard redox potential of laccases is defined as the potential of the T1 copper center, as described in Mot AC, Silaghi-Dumitrescu R., Laccases: complex architectures for one-electron oxidations, Biochemistry (Mose). 2012 December; 77(12): 1395-407. The redox potential should be less than about 460 mV, in order to be classified as “low” as contemplated herein. A common method for determining the redox potential is described in the disclosure from Xu et al., 1996: “A study of a series of recombinant fungal laccases and bilirubin oxidase that exhibit significant differences in redox potential, Substrate specificity and stability”, Biochimica et Biophysica Acta 1292, 303-311.

Particularly preferred as contemplated herein are laccases that have the consensus sequence HCHx(3)Hx(4)M, wherein x stands for “any amino acid” and the number in parentheses that follows the x sets forth the number of said amino acid(s).

Laccases as contemplated herein that are especially preferred are those that comprise an amino acid sequence that is at least 70%, and increasingly preferably at least 75%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, and 99% identical over the entire length thereof to the amino acid sequence set forth in SEQ ID NO. 1 or SEQ ID NO. 2.

SEQ ID NO. 1 is the sequence of a laccase from B. licheniformis, which comprises 513 amino acids.

SEQ ID NO. 2 is the sequence of a laccase from Streptomyces sviceus, which comprises 325 amino acids.

The identity of nucleic acid or amino acid sequences is determined by sequence comparison. This sequence comparison is based on the BLAST algorithm, which is established and commonly used in the prior art (see, for example, Altschul, S. F., Gish, W., Miller, W., Myers, E. W. & Lipman, D J. (1990) “Basic local alignment search tool.” J. Mol. Biol. 215:403-410, and Altschul, Stephan F., Thomas L. Madden, Alejandro A. Schaffer, Jinghui Zhang, Hheng Zhang, Webb Miller, and David J. Lipman (1997): “Gapped BLAST and PSI-BLAST: a new generation of protein database search programs”; Nucleic Acids Res., 25, S.3389-3402), and is done principally by associating together similar sequences of nucleotides or amino acids in the nucleic acid or amino acid sequences. Tabular mapping of the relevant positions is referred to as alignment. Another algorithm available in the prior art is the FASTA algorithm. Sequence comparisons (alignments)—in particular, multiple sequence comparisons—are created with computer programs. Commonly-used examples include the Clustal series (see, for example, Chenna et al. (2003): Multiple sequence alignment with the Clustal series of programs. Nucleic Acid Research 31, 3497-3500), T-Coffee (see, for example, Notredame et al. (2000): T-Coffee: A novel method for multiple sequence alignments. J. Mol. Biol. 302, 205-217), or programs that are based on these programs or algorithms. In the present patent application, all sequence comparisons (alignments) were created with the computer program Vector NTI® Suite 10.3 (Invitrogen Corporation, 1600 Faraday Avenue, Carlsbad, Calif., USA) with the predefined standard parameters, of which the AlignX module for sequence comparisons is based on ClustalW.

Such a comparison also allows for a report about the similarity of the compared sequences to one another. The similarity is usually indicated in percent identity, i.e., the proportion of nucleotides or amino acid residues therein or in an alignment of positions corresponding to one another. The broader concept of homology refers, in the case of amino acid sequences, to conserved amino acid substitutions, i.e., amino acids that have similar chemical activity because said amino acids exert mostly similar chemical activities within the protein. Hence, the similarity of the compared sequences can also be indicated by percent homology or percent similarity. Identity and/or homology can be indicated over entire polypeptides or genes, or only over individual regions. Homologous or identical regions of different nucleic acid or amino acid sequences are thus defined by matches in the sequences. Such regions often have identical functions. Such regions may be small and include only a small number of nucleotides or amino acids. Often, such small regions perform functions that are essential for the overall activity of the protein. It may therefore be practical to indicate sequence matches only over individual—optionally, small—regions. Unless otherwise specified, however, identities or homologies given in the present patent application refer to the total length of the respectively-indicated nucleic acid or amino acid sequence.

The laccases that can be used in the detergent as contemplated herein can be obtained from plants, fungi, and, preferably, bacteria, in particular from Bacilli and actinomycetes. Natural production of laccases is often in very low quantities. It may therefore be practical to increase production by expressing laccase genes in foreign production hosts.

To do so, it is common to use vectors that contain a nucleic acid that codes for a laccase that can be used as contemplated herein.

This may entail DNA or RNA molecules. Said molecules may be a single strand, a single strand complementary to this single strand, or a double strand. With DNA molecules in particular, sequences of the two complementary strands in each of all three possible reading frames should be taken into account. It should also be taken into account that different codons, i.e., base triplets, can code for the same amino acids, so that a given amino acid sequence can be coded by a plurality of different nucleic acids. A person skilled in the art would be capable of determining these nucleic acid sequences beyond a doubt, because—despite the degeneracy of the genetic code—defined amino acids are to be attributed to individual codons. Hence, by starting from an amino acid sequence, a person skilled in the art would have no difficulty in determining the nucleic acids coding for this amino acid sequence. Moreover, with nucleic acids, one or more codons can be replaced with synonymous codons. This aspect refers, in particular, to the heterologous expression of the enzymes that can be used as contemplated herein. Thus, every organism—for example, a host cell of a production strain—has a certain codon usage. Codon usage refers here to the translation of the genetic code into amino acids by the respective organism. Bottlenecks in protein biosynthesis can occur if the codons located on the nucleic acid are faced in the organism with a comparatively small number of loaded tRNA molecules. Although it codes for the same amino acid, the result is that a codon is translated less efficiently in the organism than a synonymous codon coding for the same amino acid. Because of the presence of a larger number of tRNA molecules for the synonymous codon, the latter can be translated more efficiently in the organism.

Using methods commonly known today such as, for example, chemical synthesis or the polymerase chain reaction (PCR) in combination with standard methods of molecular biology or protein chemistry, a person skilled in the art would be capable of preparing, on the basis of known DNA sequences and/or amino acid sequences, the corresponding nucleic acids up to complete genes. Such methods are known, for example, from Sambrook, J., Fritsch, E. F. and Maniatis, T. 2001. Molecular cloning: a laboratory manual, 3. Edition Cold Spring Laboratory Press.

Vectors within the meaning of the present invention are understood to be elements made up of nucleic acids that contain, as a characterizing nucleic acid region, a nucleic acid coding for a laccase that can be used as contemplated herein. They make it possible to establish said nucleic acid as a stable genetic element in a species or a cell line over a plurality of generations or cell divisions. In particular, when used in bacteria, vectors are special plasmids, i.e., circular genetic elements. Within the scope of the present disclosure, a nucleic acid coding for a laccase that can be used as contemplated herein is cloned into a vector. The vectors include, for example, those originating from bacterial plasmids, viruses, or bacteriophages, or predominantly synthetic vectors or plasmids having elements of very diverse origin. With the further genetic elements present in each case, vectors are capable of establishing themselves as stable units in the relevant host cells over a plurality of generations. They can be present extrachromosomally as separate units or be integrated into a chromosome or into chromosomal DNA.

Expression vectors comprise nucleic acid sequences that enable them to replicate in the host cells containing them, preferably microorganisms, especially preferably bacteria, and to express there a nucleic acid contained therein. The expression is influenced, in particular, by the promoter(s) that regulate transcription. In principle, the expression can occur by the natural promoter, which is originally localized before the nucleic acid to be expressed, but also by a host cell promoter provided on the expression vector or even by a modified or completely different promoter of a different organism or a different host cell. In the present case, at least one promoter is provided for the expression of a nucleic acid coding for a laccase that can be used as contemplated herein, and used for the expression thereof. Expression vectors can furthermore be regulatable, for example, by a change in culturing conditions or when the host cells containing them reach a specific cell density, or by the addition of specific substances, in particular, activators of gene expression. One example of such a substance is the galactose derivative isopropyl-β-D-thiogalactopyranoside (IPTG), which is used as an activator of the bacterial lactose operon (lac operon). In contrast to expression vectors, the contained nucleic acid is not expressed in cloning vectors.

A nucleic acid coding for a laccase that can be used as contemplated herein or a vector that contains such a nucleic acid is preferably transformed into a microorganism that then serves as a host cell. Alternatively, individual components, i.e., nucleic acid parts or fragments of a nucleic acid coding for a laccase that can be used as contemplated herein, can be also be introduced into a host cell in such a manner that the then-resulting host cell contains such a nucleic acid or such a vector. This procedure is especially suitable when the host cell already contains one or more constituents of such a nucleic acid or such a vector, and the further constituents are then correspondingly supplemented. Cell transformation methods are established in the prior art and are sufficiently known to a person skilled in the art. All cells—i.e., prokaryotic or eukaryotic cells—are suitable in principle as host cells. Host cells that can be advantageously manipulated genetically, for example, as regards the transformation using the nucleic acid or vector and the stable establishment thereof, are preferred, for example, single-celled fungi or bacteria. Further, preferred host cells are notable for being readily manipulated in microbiological and biotechnological terms. This refers, for example, to easy culturability, high growth rates, low demands for fermentation media, and good production and secretion rates for foreign proteins. Preferred host cells as contemplated herein secrete the (transgenically) expressed protein into the medium surrounding the host cells. Furthermore, the laccases can be modified after their production by the cells producing them, for example, by the addition of sugar molecules, formylations, aminations, etc. Post-translational modifications of this kind can functionally influence the laccases.

Host cells that are especially suitable for the production of laccases that can be used as contemplated herein are those that have activity that can be regulated on the basis of genetic regulation elements that are provided, for example, on the vector, but can also be present at the outset in these cells. They can be stimulated to expression, for example, by the controlled addition of chemical compounds serving as activators, by modifying the culturing conditions, or when a specific cell density is reached. This makes possible an economic production of the proteins that can be used as contemplated herein. One example of such a compound is IPTG, as described above.

Preferred host cells can be prokaryotic or bacterial cells. Bacteria are notable for short generation times and few demands in terms of culturing conditions. As a result, cost-effective culturing methods or production methods can be established. In addition, a person skilled in the art would have broad experience in fermentation technology, in the case of bacteria. Gram-negative or Gram-positive bacteria may be suitable for a specific production, for diverse reasons to be determined experimentally in the individual case, such as nutrient sources, product formation rate, time requirement, etc.

In gram-negative bacteria such as, for example, Escherichia coli, a plurality of proteins are secreted into the periplasmic space, i.e., into the compartment between the two membranes enclosing the cell. This can be advantageous for specific applications. Further, gram-negative bacteria can also be configured so that they discharge the expressed proteins not only into the periplasmic space but into the medium surrounding the bacterium. Gram-positive bacteria, on the other hand, such as, for example, Bacilli or actinomycetes, or other representatives of the Actinomycetales, possess no external membrane, so that secreted proteins are delivered immediately into the medium—as a rule, the nutrient medium—surrounding the bacteria, from which medium the expressed proteins can be purified. They can be isolated directly from the medium or processed further. In addition, gram-positive bacteria are related or identical to most source organisms for technically important enzymes, and usually themselves form comparable enzymes, so that they possess similar codon usage and their protein synthesis apparatus is naturally correspondingly directed.

Host cells described herein can be modified in terms of their requirements for culture conditions, can comprise other or additional selection markers, or can also express other or additional proteins. They can also be, in particular, host cells that transgenically express a plurality of proteins or enzymes.

The present disclosure can be used in principle for all microorganisms, in particular for all fermentable microorganisms, and has the result that laccases that can be used as contemplated herein can be produced by the use of such microorganisms.

Particularly preferred host cells for extracting the laccases that can be used as contemplated herein are bacteria, in particular, those selected from the genera Escherichia, Klebsiella, Bacillus, Staphylococcus, Corynebakterium, Arthrobacter, Streptomyces, Stenotrophomonas, and Pseudomonas—in particular, Escherichia coli, Klebsiella planticola, Bacillus licheniformis, Bacillus lentus, Bacillus amyloliquefaciens, Bacillus subtilis, Bacillus alcalophilus, Bacillus globigii, Bacillus gibsonii, Bacillus clausii, Bacillus halodurans, Bacillus pumilus, Staphylococcus carnosus, Corynebacterium glutamicum, Arthrobacter oxidans, Streptomyces lividans, Streptomyces coelicolor, and Stenotrophomonas maltophilia.

The host cell can also be a eukaryotic cell, however, which is characterized in that it possesses a cell nucleus. In contrast to prokaryotic cells, eukaryotic cells are capable of post-translationally modifying the formed protein. Examples thereof are fungi such as actinomycetes, or yeasts such as Saccharomyces or Kluyveromyces. This may be especially advantageous, for example, if the proteins are to undergo specific modifications, enabled by such systems, in connection with their synthesis. Modifications that eukaryotic systems carry out particularly in conjunction with protein synthesis include, for example, the bonding of low-molecular-weight compounds such as membrane anchors or oligosaccharides. Oligosaccharide modifications of this kind can be desirable, for example, in order to lower the allergenicity of an expressed protein. Co-expression with the enzymes naturally formed by such cells, for example, cellulases or lipases, can also be advantageous. Thermophilic fungal expression systems, for example, can furthermore be particularly suitable for the expression of temperature-resistant proteins or variants.

The host cells are cultured and fermented in a conventional manner, for example, in discontinuous or continuous systems. In the former case, a suitable nutrient medium is inoculated with the host cells, and the product is harvested from the medium after a period of time to be determined experimentally. Continuous fermentations are notable for the achievement of a flow equilibrium in which, over a comparatively long time period, cells die off in part but also regrow, and the formed protein can be removed simultaneously from the medium.

Fermentation methods are known from the prior art and represent the actual industrial-scale production step, generally followed by a suitable purification method for the produced product, for example, a laccase that can be used as contemplated herein.

Fermentation methods which are characterized in that fermentation is carried out via an inflow strategy are particularly appropriate. In this case, the media constituents consumed during continuous culturing are fed in. Considerable increases both in cell density and in cell mass or dry mass and/or especially in the activity of the laccase of interest can be achieved in this manner. Further, the fermentation can also be configured so that undesirable metabolic products are filtered out or are neutralized by the addition of a buffer or suitable counterions.

The produced laccase can be harvested from the fermentation medium. A fermentation method of this kind is preferred over isolation of the laccase from the host cell, i.e., product preparation from the cell mass (dry mass), but requires the provision of suitable host cells or one or more suitable secretion markers or mechanisms and/or transport systems, so that the host cells secrete the laccase into the fermentation medium. Alternatively, without secretion, the laccase can be isolated from the host cell, i.e., purification thereof from the cell mass, for example, with the use of conventional methods in enzyme chemistry such as salt precipitation, ultrafiltration, ion exchange chromatography, and hydrophobic interaction chromatography. The purification can be monitored by SDS polyacrylamide gel electrophoresis. The enzyme activity of the purified enzyme at various temperatures and pH values can be determined; similarly, the molecular weight and the isoelectric point can be determined.

Surprisingly, it has been found that only laccases that have a low redox potential are suitable as DTIs in detergents. Laccases that have an average or high redox potential do not exhibit the desired DTI effect in detergents, and also often lead to darkening and thus intensification of stains, which, of course, is undesirable.

The concentration of the laccases in the detergent as contemplated herein is preferably adjusted so that the laccase concentration in the washing liquor is in the range of 0.01 to 10 U/mL, in particular, in the range of 0.1 to 5 U/mL.

The detergent as contemplated herein preferably can be used in the temperature range of about 5° C. to about 95° C., preferably about 20° C. to about 60° C., and especially preferably about 30° C. to about 40° C.

The detergent as contemplated herein may contain additional mediators in order to more efficiently oxidize the dyes in the solution. Mediators that are suitable as contemplated herein are, for example, Tempo (2,2,6,6-tetramethyl-1-piperidinyloxy), HBT (1-hydroxybenzotriazole), ABTS (2,2′-azinobis-3-ethylbenzthiazole-6-sulphonate), NHA (N-hydroxy-acetanilide), 2,5-xylidine, ethanol, copper, 4-methylcatechol, N-hydroxyphthalimide, gallic acid, tannic acid, quercetin, syringic acid, guaiacol, dimethoxybenzyl alcohol, phenol, violuric acid (isonitro barbituric acid), phenol red, bromophenol blue, cellulose, p-coumaric acid, rooibos, o-cresol, dichloroindophenol, hydroxybenzotriazole, cycloheximide, or vanillin.

Another aspect of the present disclosure is the use of laccases to prevent or at least reduce the transfer of textile dyes from dyed textiles to undyed or differently-colored textiles when these textiles are all washed together, in particular, in surfactant-containing aqueous solutions. Particularly preferred laccases are the laccases described for the first aspect of the present disclosure.

The prevention of the staining of white textiles or differently-colored textiles by dyes washed out of textiles is particularly pronounced. The dye transfer-inhibiting laccases make a double contribution to the color constancy here, i.e., they prevent both discoloration and fading, though the effect of the prevention of staining—in particular, during washing of white textiles—is most pronounced. Another aspect of the present disclosure is therefore the use of the aforementioned laccases to prevent alteration of the color impression of textiles when the textiles are washed, in particular, in surfactant-containing aqueous solutions.

A change in color impression is in no way intended to mean the difference between the soiled and the clean textile but rather a difference in color in the clean textile before and after washing.

Another aspect of the present disclosure is a method for washing dyed textiles in surfactant-containing aqueous solutions, the method being characterized by the use of a surfactant-containing aqueous solution that contains at least one dye transfer-inhibiting laccase. The method in its simplest form is realized in that textiles requiring cleaning are brought into contact with the aqueous liquor, wherein a conventional washing machine is used or the washing can be performed by hand. It is possible in a method of this type to wash white or undyed textiles together with the dyed textile, wherein staining of the white or undyed textile is largely but not completely prevented. As contemplated herein, the method is preferably to be performed under intensive ventilation of the washing liquor, as is the case in the use of a standard household machine wash cycle.

In addition to the aforementioned dye transfer-inhibiting laccases, a detergent may contain common ingredients that are compatible with this component. Thus, it can contain, for example, in addition a further dye transfer inhibitor, preferably in amounts from about 0.1 to about 2 wt %, in particular, about 0.2 to about 1 wt %, which in a preferred embodiment is selected from the polymers of vinylpyrrolidone, vinylimidazole, and vinylpyridine-N-oxide, or the copolymers thereof. Usable are both polyvinylpyrrolidones with molecular weights from about 15,000 g/mol to about 50,000 g/mol and polyvinylpyrrolidones with higher molecular weights of, for example, up to over 1,000,000 g/mol, particularly from about 1,500,000 g/mol to about 4,000,000 g/mol, N-vinylimidazole/N-vinylpyrrolidone copolymers, polyvinyloxazolidones, copolymers based on vinyl monomers and carboxylic acid amides, pyrrolidone group-containing polyesters and polyamides, grafted polyamidoamines and polyethylenimines, polyamine-N-oxide polymers, and polyvinyl alcohols. However, enzymatic systems comprising a peroxidase and hydrogen peroxide or a substance yielding hydrogen peroxide in water can also be used. The addition of a mediator compound for peroxidase, for example, an acetosyringone, a phenol derivative, or a phenothiazine or phenoxazine, is preferred in this case, wherein the aforementioned polymeric dye transfer inhibitor active ingredients can also be used in addition. Polyvinylpyrrolidone preferably has an average molar mass in the range from about 10,000 g/mol to about 60,000 g/mol, in particular, in the range from about 25,000 g/mol to about 50,000 g/mol. Of the copolymers, those composed of vinylpyrrolidone and vinylimidazole in a molar ratio of about 5:1 to about 1:1 and having an average molar mass in the range from about 5000 g/mol to about 50,000 g/mol, particularly about 10,000 g/mol to about 20,000 g/mol, are preferred. In preferred embodiments, the detergents are free of such additional dye transfer inhibitors, however.

Detergents, which can be, in particular, present as powdered solids, in consolidated particle form, in granular form, as homogeneous solutions or suspensions, may contain in principle all known ingredients typical in such detergents, in addition to the laccases used as contemplated herein. The detergents as contemplated herein may contain, in particular, builder substances, surface-active surfactants, bleaching agents based on organic and/or inorganic peroxygen compounds, bleach activators, water-miscible organic solvents, enzymes, sequestering agents, electrolytes, pH regulators, and further auxiliaries such as optical brighteners, graying inhibitors, foam regulators, dyes, and fragrances.

The detergents preferably contain one or more surfactants, wherein in particular anionic surfactants, nonionic surfactants, and mixtures thereof, but also cationic, zwitterionic, and amphoteric surfactants are suitable.

Suitable nonionic surfactants are, in particular, alkyl glycosides and ethoxylation and/or propoxylation products of alkyl glycosides or linear or branched alcohols each having 12 to 18 C atoms in the alkyl part and 3 to 20, preferably 4 to 10 alkyl ether groups. Furthermore, corresponding ethoxylation and/or propoxylation products of N-alkylamines, vicinal diols, fatty acid esters, and fatty acid amides, which in terms of the alkyl part correspond to the cited long-chain alcohol derivatives, and of alkyl phenols having 5 to 12 C atoms in the alkyl group can be used.

Preferred nonionic surfactants are alkoxylated, advantageously ethoxylated, more particularly primary alcohols preferably containing 8 to 18 carbon atoms and an average of 1 to 12 moles of ethylene oxide (EO) per mole of alcohol, in which the alcohol radical may be linear or, preferably, 2-methyl-branched or may contain linear and methyl-branched radicals in the form of the mixtures typically present in oxoalcohol radicals. However, alcohol ethoxylates containing linear radicals of alcohols of native origin with 12 to 18 carbon atoms, for example, coconut oil alcohol, palm oil alcohol, tallow alcohol or oleyl alcohol, and an average of 2 to 8 EO per mole of alcohol are particularly preferred. Preferred ethoxylated alcohols include, for example, C₁₂-C₁₄ alcohols containing 3 EO or 4 EO, C₉-C₁₁ alcohols containing 7 EO, C₁₃-C₁₅ alcohols containing 3 EO, 5 EO, 7 EO or 8 EO, C₁₂-C₁₈ alcohols containing 3 EO, 5 EO or 7 EO, and mixtures thereof, such as mixtures of C₁₂-C₁₄ alcohol containing 3 EO and C₁₂-C₁₈ alcohol containing 7 EO. The degrees of ethoxylation mentioned are statistical mean values which, for a special product, may be either a whole number or a broken number. Preferred alcohol ethoxylates have a narrow homolog distribution (narrow range ethoxylates, NRE). In addition to these nonionic surfactants, fatty alcohols containing more than 12 EO may also be used. Examples of such fatty alcohols are (tallow) fatty alcohols containing 14 EO, 16 EO, 20 EO, 25 EO, 30 EO or 40 EO. In detergents for use in machine methods in particular, extremely low-foaming compounds are conventionally used. These preferably include C₁₂-C₁₈ alkyl polyethylene glycol polypropylene glycol ethers having up to 8 mol of ethylene oxide and propylene oxide units, each, in the molecule. However, other known, low-foaming, nonionic surfactants can also be used such as C₁₂-C₁₈ alkyl polyethylene glycol polybutylene glycol ethers having up to 8 mol of ethylene oxide and butylene oxide units, each, in the molecule, as well as end group-terminated alkyl polyalkylene glycol mixed ethers. Hydroxyl-group-containing alkoxylated alcohols, known as hydroxy mixed ethers, are also particularly preferred. The nonionic surfactants also include alkyl glycosides of the general formula RO(G)x, in which R is a primary straight-chain or methyl-branched aliphatic residue, particularly one methyl-branched in the 2-position, having 8 to 22, preferably 12 to 18 C atoms, and G is a glycose unit having 5 or 6 C atoms, preferably glucose. The degree of oligomerization x, which indicates the distribution of monoglycosides and oligoglycosides, is any number—which, as a quantity determined by analysis, can also assume fractional values—between 1 and 10; x is preferably 1.2 to 1.4 Also suitable are polyhydroxy fatty acid amides of formula

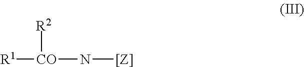

in which: R¹CO denotes an aliphatic acyl residue with 6 to 22 carbon atoms; R² denotes hydrogen, or an alkyl or hydroxyalkyl residue with 1 to 4 carbon atoms; and [Z] denotes a linear or branched polyhydroxyalkyl residue with 3 to 10 carbon atoms and 3 to 10 hydroxyl groups.

Polyhydroxy fatty acid amides are preferably derived from reducing sugars with 5 or 6 carbon atoms, in particular, from glucose. The group of polyhydroxy fatty acid amides also includes compounds of formula

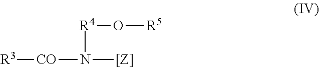

in which: R³ is a linear or branched alkyl or alkenyl residue with 7 to 12 carbon atoms; R⁴ is a linear, branched, or cyclic alkylene residue or an arylene residue with 2 to 8 carbon atoms; R⁵ is a linear, branched or cyclic alkyl residue or an aryl residue or an oxyalkyl residue with 1 to 8 carbon atoms, C₁-C₄ alkyl or phenyl residues being preferred; and [Z] is a linear polyhydroxyalkyl residue, wherein the alkyl chain thereof is substituted with at least two hydroxyl groups, or alkoxylated, preferably ethoxylated or propoxylated, derivatives of this residue. [Z] is also preferably obtained by reductive amination of a sugar such as glucose, fructose, maltose, lactose, galactose, mannose, or xylose. N-alkoxy- or N-aryloxy-substituted compounds may then, for example, be converted into desired polyhydroxy fatty acid amides by reaction with fatty acid methyl esters in the presence of an alkoxide as catalyst. A further class of preferably usable nonionic surfactants, which may be used either as sole nonionic surfactant or in combination with other nonionic surfactants, in particular together with alkoxylated fatty alcohols and/or alkylglycosides, comprises alkoxylated, preferably ethoxylated or ethoxylated and propoxylated fatty acid alkyl esters, preferably with 1 to 4 carbon atoms in the alkyl chain, in particular fatty acid methyl esters. Nonionic surfactants of the amine oxide type, for example N-coconut alkyl-N,N-dimethylamine oxide and N-tallow alkyl-N,N-dihydroxyethylamine oxide, and of the fatty acid alkanolamide type may also be suitable. The quantity of these nonionic surfactants preferably amounts to no more than that of the ethoxylated fatty alcohols, in particular no more than half the quantity thereof. So-called “gemini” surfactants may also be considered as further surfactants. These are generally taken to mean such compounds as have two hydrophilic groups per molecule. These groups are generally separated from one another by a so-called “spacer”. This spacer is generally a carbon chain, which should be long enough for the hydrophilic groups to be sufficiently far apart that they can act mutually independently. Such surfactants are in general distinguished by an unusually low critical micelle concentration and the ability to bring about a great reduction in the surface tension of water. In exceptional cases, the term “gemini surfactants” includes not only such “dimeric” surfactants, but also corresponding “trimeric” surfactants. Suitable gemini surfactants are, for example, sulfated hydroxy mixed ethers or dimer alcohol bis- and trimer alcohol tris-sulfates and -ether sulfates. End group-terminated dimeric and trimeric mixed ethers are distinguished in particular by their di- and multifunctionality. The stated end group-terminated surfactants accordingly exhibit good wetting characteristics and are low-foaming, such that they are particularly suitable for use in machine washing or cleaning methods. Gemini polyhydroxy fatty acid amides or poly-polyhydroxy fatty acid amides may, however, also be used.

Suitable anionic surfactants are, in particular, soaps and those which contain sulfate or sulfonate groups. Useful surfactants of the sulfonate type are preferably C₉-C₁₃ alkylbenzene sulfonates, olefin sulfonates, i.e. mixtures of alkene- and hydroxyalkane sulfonates, and also disulfonates, as obtained, for example, from C₁₂-C₁₈ monoolefins having terminal or internal double bonds by sulfonation with gaseous sulfur trioxide and subsequent alkaline or acidic hydrolysis of the sulfonation products. Also suitable are alkanesulfonates which are obtained from C₁₂-C₁₅ alkanes, for example by sulfochlorination or sulfoxidation with subsequent hydrolysis or neutralization. Also suitable are the esters of α-sulfo fatty acids (ester sulfonates), for example the α-sulfonated methyl esters of hydrogenated coconut, palm kernel or tallow fatty acids, which are prepared by α-sulfonation of the methyl esters of fatty acids of vegetable and/or animal origin having from 8 to 20 C atoms in the fatty acid molecule and subsequent neutralization to form water-soluble mono-salts. Preference is given to the α-sulfonated esters of hydrogenated coconut, palm, palm kernel or tallow fatty acids, it also being possible for sulfonation products of unsaturated fatty acids, for example oleic acid, to be present in small amounts, preferably in amounts not exceeding about 2 to 3 wt %. Special preference is given to α-sulfo fatty acid alkyl esters that have an alkyl chain of no more than 4 C atoms in the ester group, for example methyl esters, ethyl esters, propyl esters, and butyl esters. The use of methyl esters of α-sulfo fatty acids (MES), and also saponified di-salts thereof, is especially advantageous. Further suitable anionic surfactants are sulfonated fatty acid glycerol esters comprising mono-, di- and tri-esters and mixtures thereof, as are obtained in the preparation by esterification of a monoglycerol with from 1 to 3 moles of fatty acid or in the trans-esterification of triglycerides with from 0.3 to 2 moles of glycerol. Alk(en)yl sulfates to which preference is given are the alkali metal salts and especially the sodium salts of sulfuric acid semi-esters of C₁₂-C₁₈ fatty alcohols, for example from coconut fatty alcohol, tallow fatty alcohol, lauryl, myristyl, cetyl or stearyl alcohol, or of C₁₀-C₂₀ oxo alcohols and semi-esters of secondary alcohols having that chain length. Also preferred are alk(en)yl sulfates of said chain length that contain a synthetic straight-chain alkyl radical produced on a petrochemical basis, which have analogous breakdown characteristics to the suitable compounds based on fat-chemical raw materials. From the point of view of washing technology, special preference is given to C₁₂-C₁₆ alkyl sulfates and C₁₂-C₁₅ alkyl sulfates and also to C₁₄-C₁₅ alkyl sulfates. Suitable anionic surfactants are also 2,3-alkyl sulfates that can be obtained as commercial products of the Shell Oil Company under the name DAN®. Also suitable are the sulfuric acid monoesters of straight-chain or branched C₇-C₂₁ alcohols ethoxylated with from 1 to 6 moles of ethylene oxide, such as 2-methyl-branched C₉-C₁₁ alcohols with, on average, 3.5 moles of ethylene oxide (EO) or C₁₂-C₁₈ fatty alcohols with from 1 to 4 EO. The preferred anionic surfactants also include the salts of alkyl sulfosuccinic acid, which can also be referred to as sulfosuccinates or sulfosuccinic acid esters and which are monoesters and/or diesters of sulfosuccinic acid with alcohols, preferably fatty alcohols and, especially, ethoxylated fatty alcohols. Preferred sulfosuccinates contain C₈ to C₁₈ fatty alcohol radicals or mixtures thereof. Especially preferred sulfosuccinates contain a fatty alcohol radical derived from ethoxylated fatty alcohols that, when considered on their own, constitute nonionic surfactants. Again, special preference is given to sulfosuccinates in which the fatty alcohol radicals are derived from ethoxylated fatty alcohols having a restricted homologue distribution. It is likewise also possible to use alk(en)yl succinic acid having preferably from 8 to 18 carbon atoms in the alk(en)yl chain or salts thereof. Further anionic surfactants that come into consideration are fatty acid derivatives of amino acids, for example of N-methyltaurine (taurides) and/or of N-methylglycine (sarcosides). Special preference is given to the sarcosides and sarcosinates and, of those, more especially, to sarcosinates of higher and optionally mono- or poly-unsaturated fatty acids such as oleyl sarcosinate. Further anionic surfactants that come into consideration are, especially, soaps. Saturated fatty acid soaps such as the salts of lauric acid, myristic acid, palmitic acid, stearic acid, hydrogenated erucic acid and behenic acid and especially soap mixtures derived from natural fatty acids, for example coconut, palm kernel or tallow fatty acids, are especially suitable. Known alkenyl succinic acid salts may also be used together with these soaps or as a substitute for soaps.

The anionic surfactants, including the soaps, may be present in the form of the sodium, potassium or ammonium salts thereof and as soluble salts of organic bases, such as mono-, di- or triethanolamine. The anionic surfactants are preferably present in the form of their sodium or potassium salts, especially in the form of the sodium salts. Surfactants are present in detergents in quantities of normally about 1 to about 50 wt %, in particular, about 5 to about 30 wt %.

A detergent preferably contains at least one water-soluble and/or water-insoluble, organic and/or inorganic builder. The water-soluble organic builder substances include polycarboxylic acids, in particular citric acid and sugar acids, monomeric and polymeric aminopolycarboxylic acids, in particular glycine diacetic acid, methyl glycine diacetic acid, nitrilotriacetic acid, iminodisuccinates such as ethylenediamine-N,N′-disuccinic acid and hydroxyiminodisuccinates, ethylenediaminetetraacetic acid, as well as polyaspartic acid, polyphosphonic acids, in particular aminotris(methylenephosphonic acid), ethylenediaminetetrakis(methylenephosphonic acid), lysine tetra(methylene phosphonic acid), and 1-hydroxyethane-1,1-diphosphonic acid, polymeric hydroxy compounds such as dextrin and polymeric (poly)carboxylic acids, in particular the polycarboxylates obtainable by oxidation of polysaccharides, polymeric acrylic acids, methacrylic acids, maleic acids, and mixed polymers thereof, which may also contain small proportions of polymerizable substances without a carboxylic acid functionality incorporated therein by polymerization. The average relative molecular mass of the homopolymers of unsaturated carboxylic acids is in general between about 5,000 g/mol and about 200,000 g/mol, and that of the copolymers between about 2,000 g/mol and about 200,000 g/mol, preferably about 50,000 g/mol to about 120,000 g/mol, in each case based on free acid. One particularly preferred acrylic acid/maleic acid copolymer has an average relative molecular mass of about 50,000 to about 100,000. Suitable, albeit less preferred compounds of this class are copolymers of acrylic acid or methacrylic acid with vinyl ethers, such as vinyl methyl ethers, vinyl esters, ethylene, propylene, and styrene, the acid fraction of which amounts to at least 50 wt %. Terpolymers containing as monomers two unsaturated acids and/or the salts thereof and, as a third monomer, vinyl alcohol and/or a vinyl alcohol derivative or a carbohydrate may also be used as water-soluble organic builder substances. The first acidic monomer or the salt thereof is derived from a monoethylenically unsaturated C₃-C₈ carboxylic acid and preferably from a C₃-C₄ monocarboxylic acid, in particular from (meth)acrylic acid. The second acidic monomer or the salt thereof may be a derivative of a C₄-C₈ dicarboxylic acid, maleic acid being particularly preferred. The third monomer unit in this case is formed by vinyl alcohol and/or preferably an esterified vinyl alcohol. Vinyl alcohol derivatives which represent an ester of short-chain carboxylic acids, for example, of C₁-C₄ carboxylic acids, with vinyl alcohol, are especially preferred. Preferred polymers in this case contain about 60 to about 95 wt %, particularly about 70 to about 90 wt % of (meth)acrylic acid or (meth)acrylate, especially preferably acrylic acid or acrylate, and maleic acid or maleate, and about 5 to about 40 wt %, preferably about 10 to about 30 wt % of vinyl alcohol and/or vinyl acetate. Very especially preferred in this case are polymers in which the weight ratio of (meth)acrylic acid or (meth)acrylate to maleic acid or maleate is between about 1:1 and about 4:1, preferably between about 2:1 and about 3:1, and particularly about 2:1 and about 2.5:1. In this case, both the amounts and the weight ratios are based on the acids. The second acidic monomer or salt thereof may also be a derivative of an allyl sulfonic acid, which is substituted in the 2-position with an alkyl group, preferably with a C₁-C₄ alkyl group, or an aromatic group, derived preferably from benzene or benzene derivatives. Preferred terpolymers in this case contain about 40 to about 60 wt %, particularly about 45 to about 55 wt % of (meth)acrylic acid or (meth)acrylate, especially preferably acrylic acid or acrylate, about 10 to about 30 wt % preferably about 15 to about 25 wt % of methallyl sulfonic acid or methallyl sulfonates, and—as the third monomer—about 15 to about 40 wt %, preferably about 20 to about 40 wt % of a carbohydrate. Said carbohydrate in this case may be, for example, a mono-, di-, oligo-, or polysaccharide, mono-, di-, or oligosaccharides being preferred. Saccharose is especially preferred. Predetermined breaking points, which are responsible for the good biodegradability of the polymer, are presumably incorporated into the polymer by the use of the third monomer. These terpolymers generally have an average relative molecular mass between about 1,000 g/mol and about 200,000 g/mol, preferably between about 200 g/mol and about 50,000 g/mol. Further preferred copolymers are those preferably having acrolein and acrylic acid/acrylic acid salts or vinyl acetate as monomers. For the production of liquid detergents in particular, the organic builder substances can be used in the form of aqueous solutions, preferably in the form of about 30 to about 50 wt % aqueous solutions. All the cited acids are generally used in the form of their water-soluble salts, in particular their alkali salts.

Such organic builder substances can be contained if desired in amounts of up to 40 wt %, in particular up to 25 wt % and preferably from about 1 to about 8 wt %. Amounts close to the cited upper limit are preferably used in paste-form or liquid, in particular water-containing, detergents.

Polyphosphates in particular, preferably sodium triphosphate, are suitable as water-soluble inorganic builder materials. Crystalline or amorphous, water-dispersible alkali aluminosilicates in particular are used as water-insoluble inorganic builder materials, in amounts not exceeding 25 wt %, preferably from about 3 to about 20 wt %, and especially in amounts from about 5 to about 15 wt %. Of these, the crystalline sodium aluminosilicates in detergent quality are preferred, in particular zeolite A, zeolite P, and zeolite MAP, and optionally zeolite X. Amounts close to the cited upper limit are preferably used in solid, particulate detergents. Suitable aluminosilicates have in particular no particles with a particle size of more than 30 μm and are preferably composed of at least 80 wt % of particles with a size of less than 10 μm. Their calcium-binding capacity is generally in the range from about 100 to about 200 mg of CaO per gram.

Further water-soluble inorganic builder materials can be present in addition or alternatively to the cited water-insoluble aluminosilicates and alkali carbonate. These include, apart from polyphosphates, such as sodium triphosphate, particularly the water-soluble crystalline and/or amorphous alkali silicate builders. Such water-soluble inorganic builder materials are present in the detergents preferably in amounts from about 1 to about 20 wt %, particularly from about 5 to about 15 wt %. Alkali silicates that can be used as builder materials preferably have a molar ratio of alkali oxide to SiO₂ of less than 0.95, particularly from 1:1.1 to 1:12, and can be amorphous or crystalline. Preferred alkali silicates are sodium silicates, particularly amorphous sodium silicates, with a molar ratio Na₂O:SiO₂ of 1:2 to 1:2.8. Crystalline phyllosilicates of the general formula Na₂SiₓO₂ₓ₊₁.yH₂O, in which the so-called modulus x is a number from 1.9 to 4 and y is a number from 0 to 20, with preferred values for x being 2, 3, or 4, are preferably used as crystalline silicates, which can be present alone or in a mixture with amorphous silicates. Preferred crystalline phyllosilicates are those in which x assumes the values 2 or 3 in the cited general formula. In particular both β- and δ-sodium disilicates (Na₂Si₂O₅.yH₂O) are preferred. Virtually anhydrous crystalline alkali silicates of the aforementioned general formula prepared from amorphous alkali silicates, in which x denotes a number from 1.9 to 2.1, can also be used in the detergents. In a further preferred embodiment, a crystalline sodium phyllosilicate with a modulus of 2 to 3 is used, such as can be prepared from sand and soda. Sodium silicates with a modulus in the range from 1.9 to 3.5 are used in a further preferred embodiment. In a preferred embodiment of such detergents, a granular compound of alkali silicate and alkali carbonate is used, as is obtainable commercially under the name Nabion® 15, for example.

Suitable bleaching agents are those on a chlorine base, such as, in particular, alkali hypochlorite, dichloroisocyanuric acid, trichloroisocyanuric acid, and salts thereof, but particularly also those on a peroxygen base. Suitable peroxygen compounds are in particular organic peracids or peracid salts of organic acids, such as phthalimidopercaproic acid, perbenzoic acid, monoperoxyphthalic acid, and diperdodecanedioic acid, as well as salts thereof, such as magnesium monoperoxyphthalate, hydrogen peroxide, and inorganic salts which give off hydrogen peroxide under washing conditions, such as perborate, percarbonate, and/or persilicate, and hydrogen peroxide inclusion compounds, such as H₂O₂ urea adducts. In this regard, hydrogen peroxide can also be generated with the aid of an enzymatic system, i.e., an oxidase and its substrate. If solid peroxygen compounds are to be used, they may be used in the form of powders or granules, which may also be encapsulated in a manner known in principle. Alkali percarbonate, alkali perborate monohydrate, or hydrogen peroxide in the form of aqueous solutions, containing 3 to 10 wt % of hydrogen peroxide, is used especially preferably. If a detergent contains peroxygen compounds, these are present in amounts of preferably up to 25 wt %, particularly from 1 to 20 wt %, and especially preferably from 7 to 20 wt %.

In particular, compounds, which under perhydrolysis conditions produce optionally substituted perbenzoic acid and/or aliphatic peroxocarboxylic acids a having 1 to 12 C atoms, particularly 2 to 4 C atoms, can be used alone or in mixtures as bleach-activating compounds that yield peroxocarboxylic acids under perhydrolysis conditions. Bleach activators bearing 0- and/or N-acyl groups in particular having the stated number of C atoms and/or optionally substituted benzoyl groups are suitable. Preferred are multiply acylated alkylenediamines, in particular tetraacetylethylenediamine (TAED), acylated glycolurils, in particular tetraacetylglycoluril (TAGU), acylated triazine derivatives, in particular 1,5-diacetyl-2,4-dioxohexahydro-1,3,5-triazine (DADHT), N-acylimides, in particular N-nonanoyl succinimide (NOSI), acylated phenol sulfonates or carboxylates or the sulfonic or carboxylic acids thereof, in particular n-nonanoyl- or isononanoyl- or lauryloxybenzenesulfonate (NOBS or iso-NOBS or LOBS), or decanoyloxybenzoate (DOBA), the formal carbonate derivatives thereof such as 4-(2-decanoyloxyethoxycarbonyloxyl)benzene sulfonate (DECOBS), acylated polyhydric alcohols, in particular triacetin, ethylene glycol diacetate, and 2,5-diacetoxy-2,5-dihydrofuran and acetylated sorbitol and mannitol and mixtures thereof (SORMAN), acylated sugar derivatives, in particular pentaacetyl glucose (PAG), pentaacetyl fructose, tetraacetyl xylose and octaacetyl lactose, acetylated, optionally N-alkylated glucamine and gluconolactone, and/or N-acylated lactams, for example, N-benzoylcaprolactam.

In addition to the compounds forming peroxocarboxylic acids under perhydrolysis conditions, other bleach-activating compounds may be present such as, for example, nitriles, from which perimidic acids form under perhydrolysis conditions. These include, in particular, aminoacetonitrile derivatives with a quaternized nitrogen atom according to the formula

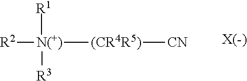

in which R¹ stands for —H, —CH₃, a C₂₋₂₄ alkyl or alkenyl group, a substituted C₁₋₂₄ alkyl or C₂₋₂₄ alkenyl group with at least one substituent from the group —Cl, —Br, —OH, —NH₂, —CN, and —N(+)—CH₂—CN, an alkyl or alkenylaryl group with a C₁₋₂₄ alkyl group, or stands for a substituted alkyl or alkenylaryl group with at least one, preferably two, optionally substituted C₁₋₂₄ alkyl group(s) and optionally further substituents on the aromatic ring; R² and R³, independently of one another, are selected from —CH₂—CN, —CH₃, —CH₂—CH₃, —CH₂—CH₂—CH₃, —CH(CH₃)—CH₃, —CH₂—OH, —CH₂—CH₂—OH, —CH(OH)—CH₃, —CH₂—CH₂—CH₂—OH, —CH₂—CH(OH)—CH₃, —CH(OH)—CH₂—CH₃, —(CH₂CH₂—O)ₙH where n=1, 2, 3, 4, 5 or 6; R⁴ and R⁵, independently of one another, have a meaning indicated above for R¹, R², or R³, wherein at least 2 of the mentioned groups, particularly R² and R³, also with inclusion of the nitrogen atom and optionally further heteroatoms can be connected together with ring closure and then preferably form a morpholino ring; and X is a charge-equalizing anion, preferably selected from benzene sulfonate, toluene sulfonate, cumol sulfonate, C₉₋₁₅ alkylbenzene sulfonates, C₁₋₂₀ alkyl sulfates, C₈₋₂₂ carboxylic acid methyl ester sulfonates, sulfate, hydrogen sulfate, and mixtures thereof, can be used. Oxygen-transferring sulfonimines and/or acylhydrazones can also be used.

The presence of bleach-catalyzing transition metal complexes is also possible. These are preferably selected from among cobalt, iron, copper, titanium, vanadium, manganese, and ruthenium complexes. Ligands suitable in such transition metal complexes are both inorganic and organic compounds, which include, apart from carboxylates, in particular, compounds with primary, secondary, and/or tertiary amine and/or alcohol functions, such as pyridine, pyridazine, pyrimidine, pyrazine, imidazole, pyrazole, triazole, 2,2′-bispyridylamine, tris-(2-pyridylmethyl)amine, 1,4,7-triazacyclononane, 1,4,7-trimethyl-1,4,7-triazacyclononane, 1,5,9-trimethyl-1,5,9-triazacyclododecane, (bis-((1-methylimidazol-2-yl)methyl))-(2-pyridylmethyl)amine, N,N′-(bis-(1-methylimidazol-2-yl)methyl)ethylenediamine, N-bis-(2-benzimidazolylmethyl)aminoethanol, 2,6-bis-(bis-(2-benzimidazolylmethyl)aminomethyl)-4-methylphenol, N,N,N′,N′-tetrakis-(2-benzimidazolylmethyl)-2-hydroxy-1,3-diaminopropane, 2,6-bis-(bis-(2-pyridylmethyl)aminomethyl)-4-methylphenol, 1,3-bis-(bis-(2-benzimidazolylmethyl)aminomethyl)benzene, sorbitol, mannitol, erythritol, adonitol, inositol, lactose, and optionally substituted salens, porphins, and porphyrins. The inorganic neutral ligands include in particular ammonia and water. If not all coordination sites of the transition metal central atom are occupied by neutral ligands, then the complex contains further, preferably anionic, and among these in particular mono- or bidentate ligands. These include in particular halides such as fluoride, chloride, bromide, and iodide, and the (NO₂)⁻ group, i.e., a nitro ligand or a nitrito ligand. The (NO₂)⁻ group can also be bound to a transition metal to form a chelate or it can bridge two transition metals asymmetrically or with η¹-O coordination. Apart from the mentioned ligands, the transition metal complexes may bear still further ligands, generally with a simpler structure, particularly mono- or polyvalent anionic ligands. Examples are nitrate, acetate, trifluoracetate, formate, carbonate, citrate, oxalate, perchlorate, and complex anions such as hexafluorophosphate. The anionic ligands are intended to provide the charge equalization between the transition metal central atom and the ligand system. Oxo ligands, peroxo ligands, and imino ligands may also be present. Such ligands in particular may also have a bridging effect so that polynuclear complexes are formed. In the case of bridged binuclear complexes, the two metal atoms in the complex need not be the same. Binuclear complexes in which the two transition metal central atoms have different oxidation numbers may also be used. In the absence of anionic ligands or if the presence of anionic ligands does not lead to charge equalization in the complex, the transition metal complex compounds to be used in accordance with the disclosure contain anionic counterions which neutralize the cationic transition metal complex. These anionic counterions include in particular nitrate, hydroxide, hexafluorophosphate, sulfate, chlorate, perchlorate, halides such as chloride, or the anions of carboxylic acids, such as formate, acetate, oxolate, benzoate, or citrate. Examples of transition metal complex compounds that may be used are Mn(IV)₂(μ-O)₃(1,4,7-trimethyl-1,4,7-triazacyclononane)dihexafluorophosphate, [N,N′-bis[(2-hydroxy-5-vinylphenyl)methylene]-1,2-diaminocyclohexane]manganese(III) chloride, [N,N′-bis[(2-hydroxy-5-nitrophenyl)methylene]-1,2-diaminocyclohexane]manganese(III) acetate, [N,N′-bis[(2-hydroxyphenyl)methylene]-1,2-phenylendiamine]manganese(III) acetate, [N,N′-bis[(2-hydroxyphenyl)methylene]-1,2-diaminocyclohexane]manganese(III) chloride, [N,N′-bis[(2-hydroxyphenyl)methylene]-1,2-diaminoethane]manganese (III) chloride, [N,N′-bis[(2-hydroxy-5-sulfonatophenyl)methylene]-1,2-diaminoethane]manganese(III) chloride, manganese oxalate complexes, nitropentaammine-cobalt(III) chloride, nitritopentaammine-cobalt(III) chloride, hexaammine-cobalt(III) chloride, chloropentaammine-cobalt(III) chloride, and the peroxo complex [(NH₃)₅Co—O—O—Co(NH₃)₅]Cl₄.

Enzymes that can be used in the detergents in addition to the aforementioned laccases are those from the class of amylases, proteases, lipases, cutinases, pullulanases, hemicellulases, cellulases, oxidases, and peroxidases, and mixtures thereof. It would also be possible as contemplated herein to use one or more additional laccases, or multicopper oxidases, in addition to the aforementioned laccases. Particularly suitable are enzymatic active substances obtained from fungi or bacteria, such as Bacillus subtilis, Bacillus licheniformis, Bacillus lentus, Streptomyces griseus, Humicola lanuginosa, Humicola insolens, Pseudomonas pseudoalcaligenes, Pseudomonas cepacia, or Coprinus cinereus. The enzymes may be adsorbed onto supports and/or encapsulated in shell-forming substances to protect them against premature inactivation. They are contained in the detergents or cleaning agents as contemplated herein preferably in amounts up to 5 wt %, in particular, from about 0.2 to about 4 wt %. If the detergent as contemplated herein contains protease, it preferably has a proteolytic activity in the range from around about 100 PE/g to around about 10,000 PE/g, in particular about 300 PE/g to about 8000 PE/g. If a plurality of enzymes are to be used in the detergent as contemplated herein, this can be carried out by incorporating the two or more separate enzymes or enzymes formulated separately in a known manner or two or more enzymes formulated together in granules.

Organic solvents that can be used in addition to water in the detergents, particularly if they are in liquid or pasty form, include alcohols having 1 to 4 C atoms, in particular, methanol, ethanol, isopropanol, and tert-butanol, diols having 2 to 4 C atoms, particularly ethylene glycol and propylene glycol, and mixtures thereof, and ethers derivable from the cited classes of compounds. Such water-miscible solvents are preferably present in the detergents as contemplated herein in amounts not exceeding 30 wt %, in particular, from about 6 to about 20 wt %.

To set a desired pH value that is not established automatically by mixing the other components, the detergents as contemplated herein may contain system-compatible and environmentally compatible acids, in particular citric acid, acetic acid, tartaric acid, malic acid, lactic acid, glycolic acid, succinic acid, glutaric acid, and/or adipic acid, but also mineral acids, in particular sulfuric acid, or bases, in particular ammonium or alkali hydroxides. Such pH regulators are contained in the detergents as contemplated herein in amounts preferably not exceeding 20 wt %, in particular, from about 1.2 to about 17 wt %.

Graying inhibitors have the task of keeping dirt—which has been dissolved out of the textile fibers—suspended in the liquor. Water-soluble colloids of a mainly organic nature are suitable for this purpose, for example, starch, size, gelatin, salts of ether carboxylic acids or ether sulfonic acids of starch or cellulose or salts of acidic sulfuric acid esters of cellulose or starch. Water-soluble polyamides containing acidic groups are also suitable for this purpose. Derivatives of starch other than those stated above, for example, aldehyde starches, may be used furthermore. Cellulose ethers, such as carboxymethylcellulose (Na salt), methylcellulose, hydroxyalkylcellulose, and mixed ethers, such as methylhydroxyethylcellulose, methylhydroxypropylcellulose, methylcarboxymethylcellulose, and mixtures thereof, are preferably used, for example, in amounts from about 0.1 to about 5 wt %, based on the detergent.

Detergents may contain as optical brighteners, for example, derivatives of diaminostilbene disulfonic acid or the alkali metal salts thereof, although they preferably contain no optical brighteners for use as a color detergent. Suitable examples are salts of 4,4′-bis(2-anilino-4-morpholino-1,3,5-triazinyl-6-amino)stilbene-2,2′-disulfonic acid or compounds of similar structure which, instead of the morpholino group, bear a diethanolamino group, a methylamino group, an anilino group, or a 2-methoxyethylamino group. Brighteners of the substituted diphenylstyryl type furthermore may be present, for example, the alkali salts of 4,4′-bis(2-sulfostyryl)diphenyl, 4,4′-bis(4-chloro-3-sulfostyryl)diphenyl, or 4-(4-chlorostyryl)-4′-(2-sulfostyryl)diphenyl. Mixtures of the aforementioned optical brighteners may also be used.

Especially for use in a machine method, it may be advantageous to add conventional foam inhibitors to the detergents. Suitable foam inhibitors are, for example, soaps of natural or synthetic origin, which have a high proportion of C₁₈-C₂₄ fatty acids. Suitable nonsurfactant foam inhibitors are, for example, organopolysiloxanes and mixtures thereof with microfine, optionally silanized silicic acid, as well as paraffins, waxes, microcrystalline waxes, and mixtures thereof with silanized silicic acid or bis-fatty acid alkylene diamides. Mixtures of different foam inhibitors are also used advantageously, for example, mixtures of silicones, paraffins, or waxes. The foam inhibitors, in particular foam inhibitors containing silicone and/or paraffin, are preferably bound to a granular carrier substance soluble or dispersible in water. Mixtures of paraffins and distearyl ethylene diamide are particularly preferred here.

The production of solid detergents presents no difficulties and may occur in a known manner, for example, by spray drying or granulation, with enzymes and other possible thermally sensitive constituents such as, for example, bleaching agents optionally being added separately later. A method having an extrusion step is preferred for producing detergents with an elevated bulk density, particularly in the range from about 650 g/L to about 950 g/L.

To produce detergents in tablet form, which may be monophasic or multiphasic, single-colored or multicolored, and in particular may consist of one layer or a plurality of layers, in particular, two layers, one preferably proceeds such that all ingredients, optionally for each layer, are mixed together in a mixer and the mixture is compressed by means of conventional tablet presses, for example, eccentric presses or rotary presses, with pressing forces in the range from approximately about 50 to about 100 kN, preferably at about 60 to about 70 kN. In particular in the case of multilayer tablets, it may be advantageous for at least one layer to be precompressed. This is preferably carried out at pressing forces between about 5 and about 20 kN, in particular at about 10 to about 15 kN. Tablets that are breaking-resistant and yet dissolve sufficiently rapidly under conditions of use and with breaking and bending strengths usually from about 100 to about 200 N, but preferably of above 150 N are easily obtained in this way. A tablet produced in this manner preferably has a weight from about 10 g to about 50 g, in particular from about 15 g to about 40 g. The shape of the tablets is arbitrary and may be round, oval, or angular, intermediate shapes also being possible. Corners and edges are advantageously rounded. Round tablets preferably have a diameter from about 30 mm to about 40 mm. In particular, the size of angular or cuboidal tablets, which are predominantly introduced by means of the dispenser of a washing machine, depends on the geometry and volume of said dispenser. Preferred embodiments have, for example, a base area of (about 20 to about 30 mm)×(about 34 to about 40 mm), in particular of about 26× about 36 mm or of about 24× about 38 mm.

Liquid or pasty detergents in the form of solutions containing conventional solvents are generally produced by simply mixing the constituents, which may be introduced into an automatic mixer in bulk or as a solution. The detergent described herein—in particular, the low-water or water-free liquid detergents—may be packaged in a water-soluble encapsulation and thus be a part of a water-soluble packaging. If the detergent is packaged in a water-soluble encapsulation, the water content is preferably less than 10 wt % in relation to the entire detergent, and the anionic surfactants—if any—are preferably present in the form of the ammonium salts thereof.

Neutralization with amines, unlike bases such as NaOH or KOH, does not lead to the formation of water. Thus, low-water detergents can be produced that are directly suitable for use in water-soluble coverings.

In addition to the detergent, a water-soluble packaging contains a water-soluble encapsulation. The water-soluble encapsulation is preferably formed by a water-soluble film material.

Such water-soluble packagings may be produced by either vertical form fill sealing (VFFS) methods or thermoforming methods.

Thermoforming generally includes forming a first layer of a water-soluble film material to produce indentations for receiving a composition, introducing the composition into the indentations, covering the indentations filled with the composition with a second layer of a water-soluble film material, and sealing the first and second layers together at least around the indentations.

The water-soluble encapsulation is preferably made of a water-soluble film material selected from the group comprising polymers or polymer blends. The envelope may be formed of one or of two or more layers of the water-soluble film material. The water-soluble film material of the first layer and further layers, if any, may be identical or different.

The water-soluble packaging containing the detergent and the water-soluble encapsulation may have one or more chambers. The liquid detergent may be contained in one or more chambers, if any are present, of the water-soluble encapsulation. The amount of liquid detergent preferably corresponds to the full or half dose that is needed for a wash cycle. It is preferred for the water-soluble encapsulation to contain polyvinyl alcohol or a polyvinyl alcohol copolymer.

Suitable water-soluble films for producing the water-soluble encapsulation are preferably based on a polyvinyl alcohol or a polyvinyl alcohol copolymer having a molecular weight in the range from about 10,000 to about 1,000,000 g/com, preferably from about 20,000 to about 500,000 g/mol, more preferably from about 30,000 to about 100,000 g/mol, and in particular from about 40,000 to about 80,000 g/mol.

Polymers selected from the group comprising acrylic acid-containing polymers, polyacrylamides, oxazoline polymers, polystyrene sulfonates, polyurethanes, polyesters, polyether polylactic acid, and/or mixtures of the above polymers, can be added to a film material that is suitable for producing the water-soluble encapsulation.

Preferred polyvinyl alcohol copolymers comprise, in addition to vinyl alcohol, dicarboxylic acids as further monomers. Suitable dicarboxylic acids are itaconic acid, malonic acid, succinic acid, and mixtures thereof, itaconic acid being preferred.

Likewise preferred polyvinyl alcohol copolymers comprise, in addition to vinyl alcohol, an ethylenically unsaturated carboxylic acid, the salt thereof, or ester thereof. Especially preferably, such polyvinyl alcohol copolymers contain acrylic acid, methacrylic acid, acrylic acid esters, methacrylic acid esters, or mixtures thereof, in addition to vinyl alcohol.

Suitable water-soluble films for use in the encapsulations of the water-soluble packagings as contemplated herein are films marketed by the company MonoSol LLC, for example, under the name M8630, C8400, or M8900. Other suitable films comprise films with the name Solublon® PT, Solublon® GA, Solublon® KC, or Solublon® KL from Aicello Chemical Europe GmbH or the films VF-HP from Kuraray.

The water-soluble packagings can have a substantially dimensionally stable spherical and pillow-shaped configuration with a circular, elliptical, square, or rectangular basic form.

The water-soluble packaging may have one or more chambers for storing one or more agents. If the water-soluble packaging has two or more chambers, at least one chamber contains the liquid detergent. The further chambers can each contain a solid or a liquid detergent.

### EXAMPLES

The following examples illustrate the present invention, but do so in a non-limiting manner.

### Example 1: Use of Two Bacterial Laccases as DTIs

Two bacterial laccases having the SEQ ID NOS: 1 and 2 were tested as follows for their suitability as DTIs. The protein region that is of importance for the redox potential is set forth hereinbelow for both laccases, wherein conserved amino acids are marked by being in bold. Most importantly, the bolded amino acid M (methionine) is of importance for low redox potential.

1)
SEQ ID NO: 1
485 GRYVWHCHILEHEDYDMMRP . . .
2)
SEQ ID NO: 2
278 GAWMYHCHVQSHSDMGMVGL . . .

DTI Test Setup:

A Staining Scale Rating (SSR), which is based on ISO 105-A04, was carried out to determine the dye transfer-inhibiting properties of the individual detergents. To this end, in batches each with a volume of 100 mL, two white fabrics (A: 6×16 cm standard cotton fabric wfk; B: 6×16 cm standard polyamide fabric) were washed for 30 minutes at 50° C. in a Linitest Plus apparatus from the company Atlas with a color former (Direct Red 83:1, Hohenstein), the concentration of which was 0.3 g/fabric swatch, with the use a commercially-available liquid detergent composition containing no dye transfer inhibitor (rate of addition 5.21 g/L) and with addition of (batch 2) 100 U of laccase 1 (SEQ ID NO:1) or (batch 3) 100 U of laccase 2 (SEQ ID NO: 2), in accordance with the Hohenstein method (analogous to ISO 105 C06), then incubated at 40 rpm/min, rinsed with water (16° DPH), and hung up to dry at room temperature. Next, the degree of discoloration of the two fabrics was determined spectrophotometrically. The same dye transfer inhibitor-free detergent composition (batch 1) was also tested in the same manner, but without the addition of laccase, for the purpose of comparison. Also for the purpose of comparison, two additional batches were tested, each with 100 U of a laccase having a high redox potential, in the same method (batch 4: Ecostone LCL 45, from the company AB Enzymes; batch 5: BioDet-BBS, from the company Biozyme).

The degree of discoloration was then specified in values of 1 (strong discoloration) to 5 (no discoloration).

| dye- | dye | | | | | |
|---|---|---|---|---|---|---|
| acceptor | donator | Batch 1 | Batch 2 | Batch 3 | Batch 4 | Batch 5 |
| cotton | Direct | 2.8 | 3.6 | 4.5 | 2.8 | 2.7 |
| | Red 83:1 | | | | | |

It is readily apparent that the two laccases exhibit a significant improvement of dye transfer from Direct Red 83:1 onto cotton (significance being defined as a change of at least 0.5 units).

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

1. A detergent, comprising at least one laccase as a dye transfer inhibitor.

2. The detergent according to claim 1, wherein the at least one laccase has a redox potential under 460 mV, and wherein the standard redox potential of the laccase is defined as the potential of the T1 copper center.

3. The detergent according to claim 1, wherein the at least one laccase is selected from laccases that have the consensus sequence HCHx(3)Hx(4)M, wherein x stands for “any amino acid” and the number in parentheses that follows the x sets forth the number of said “amino acid(s)”.

4. The detergent according to claim 1, wherein the at least one laccase is selected from laccases that comprise an amino acid sequence that is at least 70% identical over the entire length thereof to the amino acid sequence set forth in SEQ ID NO. 1 or SEQ ID NO. 2.

5. The detergent according to claim 1, wherein the concentration of the at least one laccase is adjusted so that the laccase concentration in a washing liquor is in the range of from about 0.01 to about 10 U/mL.

6. The detergent according to claim 1, wherein the detergent is usable in the temperature range of from about 5° C. to about 95° C.

7. The detergent according to claim 1, further comprising additional mediators selected from 2,2,6,6-tetramethyl-1-piperidinyloxy, 1-hydroxybenzotriazole, 2,2′-azinobis-3-ethylbenzthiazole-6-sulphonate, N-hydroxy-acetanilide, 2,5-xylidine, ethanol, copper, 4-methylcatechol, N-hydroxyphthalimide, gallic acid, tannic acid, quercetin, syringic acid, guaiacol, dimethoxybenzyl alcohol, phenol, violuric acid, phenol red, bromophenol blue, cellulose, p-coumaric acid, rooibos, o-cresol, dichloroindophenol, hydroxybenzotriazole, cycloheximide, or vanillin, and combinations thereof.

8. (canceled)

9. (canceled)

10. The detergent according to claim 1, wherein the at least one laccase is selected from laccases that comprise an amino acid sequence that is at least 90% identical over the entire length thereof to the amino acid sequence set forth in SEQ ID NO. 1 or SEQ ID NO. 2.

11. The detergent according to claim 1, wherein the at least one laccase is selected from laccases that comprise an amino acid sequence that is at least 95% identical over the entire length thereof to the amino acid sequence set forth in SEQ ID NO. 1 or SEQ ID NO. 2.

12. The detergent according to claim 1, wherein the concentration of the at least one laccase is adjusted so that the laccase concentration in a washing liquor is in the range of from about 0.1 to about 5 U/mL.

13. The detergent according to claim 1, wherein the detergent is usable in the temperature range of from about 30° C. to about 40° C.

14. The detergent according to claim 1, wherein the detergent is usable in the temperature range of from about 20° C. to about 60° C.

15. The detergent according to claim 1, further comprising an additional dye transfer inhibitor.

16. The detergent according to claim 15, wherein the additional dye transfer inhibitor is selected from the polymers of vinylpyrrolidone, vinylimidazole, and vinylpyridine-N-oxide, or the copolymers thereof, and combinations thereof.

17. A detergent, comprising:
at least one laccase as a dye transfer inhibitor;
wherein the at least one laccase has a redox potential under 460 mV;
wherein the standard redox potential of the laccase is defined as the potential of the T1 copper center; and
wherein the at least one laccase is selected from laccases that have the consensus sequence HCHx(3)Hx(4)M, wherein x stands for “any amino acid” and the number in parentheses that follows the x sets forth the number of said “amino acid(s)”.

18. The detergent according to claim 17, wherein the at least one laccase is selected from laccases that comprise an amino acid sequence that is at least 70% identical over the entire length thereof to the amino acid sequence set forth in SEQ ID NO. 1 or SEQ ID NO. 2.

19. The detergent according to claim 1, further comprising additional mediators selected from 2,2,6,6-tetramethyl-1-piperidinyloxy, 1-hydroxybenzotriazole, 2,2′-azinobis-3-ethylbenzthiazole-6-sulphonate, N-hydroxy-acetanilide, 2,5-xylidine, ethanol, copper, 4-methylcatechol, N-hydroxyphthalimide, gallic acid, tannic acid, quercetin, syringic acid, guaiacol, dimethoxybenzyl alcohol, phenol, violuric acid, phenol red, bromophenol blue, cellulose, p-coumaric acid, rooibos, o-cresol, dichloroindophenol, hydroxybenzotriazole, cycloheximide, or vanillin, and combinations thereof.

20. A method for washing dyed textiles, the method comprising the step of:
applying a detergent comprising at least one laccase as a dye transfer inhibitor to a dyed textile.

21. The method according to claim 20, further comprising the steps of:
providing the dyed textile;
providing an undyed or differently-colored textile; and
washing the dyed textile, and the undyed or differently-colored textile in the presence of the detergent to prevent or at least reduce the transfer of textile dyes from the dyed textile to the undyed or differently-colored textile.

22. The method according to claim 20, wherein the at least one laccase has a redox potential under 460 mV, and wherein the standard redox potential of the laccase is defined as the potential of the T1 copper center.


SEQUENCE LISTING (2 sequences)

SEQ ID NO: 1 - protein, 513 aa, B. licheniformis
  Laccase aus B. licheniformis
MKLEKFVDRLPIPQVLQPQSKSKEMTYYEVTMKEFQQQLHRDLPPTRLFGYNGVYPGPTFEVQKHEKVAVKWLNKLPDRHFLPVDHTLHDDGHHEHEVKTVVHLHGGCTPADSDGYPEAWYTKDFHAKGPFFEREVYEYPNEQDATALWYHDHAMAITRLNVYAGLVGLYFIRDREERSLNLPKGEYEIPLLIQDKSFHEDGSLFYPRQPDNPSPDLPDPSIVPAFCGDTILVNGKVWPFAELEPRKYRFRILNASNTRIFELYFDHDITCHQIGTDGGLLQHPVKVNELVIAPAERCDIIVDFSRAEGKTVTLKKRIGCGGQDADPDTDADIMQFRISKPLKQKDTSSLPRILRKRPFYRRHKINALRNLSLGAAVDQYGRPVLLLNNTKWHEPVTETPALGSTEIWSIINAGRAIHPIHLHLVQFMILDHRPFDIERYQENGELVFTGPAVPPAPNEKGLKDTVKVPPGSVTRIIATFAPYSGRYVWHCHILEHEDYDMMRPLEVTDVRHQ

SEQ ID NO: 2 - protein, 325 aa, Streptomyces sviceus
  Laccase aus Streptomyces sviceus
MGALDRRGFNRRVLLGGAAVATSLSLAPEARSDAGPAQAAPGGEVRRIKLYAERLADGQMGYGLEKGRATIPGPLIELNEGDTLHIEFENTMDVRASLHVHGLDYEVSSDGTTLNKSDVEPGGTRTYTWRTHAPGRRSDGTWRAGSAGYWHYHDHVVGTEHGTGGIRKGLYGPVIVRRKGDVLPDATHTIVFNDMLINNRPAHSGPNFEATVGDRVEFVMITHGEYYHTFHMHGHRWADNRTGMLTGPDDPSQVVDNKIVGPADSFGFQVIAGEGVGAGAWMYHCHVQSHSDMGMVGLFLVKKTDGTIPGYEPHEHSGQRAEHHH